(19) United States
(12) Patent Application Publication
(10) Pub. No.: US 2020/0113948 A1
(43) Pub. Date: Apr. 16, 2020

(54) A NATURAL FORMULA FOR TREATING DIGESTIVE PROBLEMS AND INFECTIONS

(71) Applicant: Ayoub Abdullah Mohammed, Salalah (OM)

(72) Inventor: Ayoub Abdullah Mohammed, Salalah (OM)

(21) Appl. No.: 16/658,146

(22) PCT Filed: Apr. 19, 2017
(86) PCT No.: PCT/OM2017/000003

§ 371 Date: Oct. 20, 2019

(51) Int. Cl. A61K 35/644; A61K 36/71; A61K 36/55; A61K 36/18; A61K 9/14

(52) U.S. Cl. CPC A61K 35/644; A61K 36/71; A61K 9/14; A61K 36/18; A61K 36/55

(57) ABSTRACT

The application relates to a natural formula of organic sedra honey, black seed, linseed and nutmeg. Mixing all these ingredients together strengthens the effectiveness of these herbs. The natural formula is used for treating digestive problems such as stomach acidity, stomach inflammation (gastritis) and inflammatory bowel syndrome.

### TECHNICAL BACKGROUND

To date, the causes of the IBD is unknown, and no specific cure is available yet. All previously invented treatments for Inflammatory Bowel Disease (IBD), ulcerative colitis and Crohn's disease disorders are not considered curable treatments. They have a temporal effect and formerly used as sedative treatments.

Unfortunately, most of these drugs and treatments are commercialized and barely effect anything other than psychological reflection in the minds of the patients. The main reason for these therapeutic defects as well as being sold as commercial product is that the drugs are not extracted from elements that have high therapeutic characteristics. Also, the major problem is that they are focusing on the marketing side more than pharmacological benefits of the drug. Therefore patients remain suffering from the digestive problems throughout their lives; although the digestive problems are not complex as evidenced by it remains for years and do not lead to devastating health consequences that end to death. This is an evidence of the simplicity of these cases and do not need to the removal of the organ but the weakness of the commercial drugs available on the market lead to the fact that the removal of the injured organ is a more useful treatment, but in fact it is not.

### SUMMARY

The formula is a natural organic compound consists of famous traditional herbs available in all kitchens around the world. These herbs are safe and healthy and each one has its own unique impact on the digestive system. Mixing all these ingredients together to produce this treatment has strengthen the effectiveness of these herbs. This formula was designed and improved to treat various digestive problems such as stomach acidity, stomach inflammation (gastritis), and Inflammatory Bowel Syndrome and has showed an effective positive impact on all digestive system organs.

### DETAILED DESCRIPTION

The effect of commonly used drugs for digestive diseases are not curable, even partially. They are sedatives and painkillers. Some of which are sold in the regular grocery and can be taken without a prescription in the sense that they are not medical drugs. As for stomach acidity, these products' effect is temporary; two hours and some of them are three hours—according to the popular television advertisements. As for gastric wall infections, the only medicine currently prescribed is antibiotics, which is usually followed by; in critical cases; removal of the injured part of the stomach. Moreover, there are no specialized treatments for inflammation and stomach wall ulcers. As for the colon, the most commonly prescribed treatments for those who suffer from it are the painkillers that have limited effect and time-effect, where the pain is returned after hours without curing the illness permanently.

To conclude, all the problems of the digestive system currently do not have effective drugs, but they are only non-specialized palnkillers. After presenting the potential toxicity and side effects of the practiced treatments, it is important to improve less invasive treatments extracted from the Mother Nature and accessible to everyone.

This formula—it is a completely safe and healthy treatment produced from herbs available in every kitchen and contained in our daily meals. These herbs in our meals have a slight therapeutic impact that people usually do not notice. This is because our meals lack the exact compositions that improve the therapeutic impact strength. Over the past 13 years of thoughtful, examined and proven experience, Hadeem has been prepared to contain the exact required compositions and is completely safe components. Each one of these elements have a particular impact and therapeutic properties related to the digestive system. Only when these ingredients were mixed thoughtfully by experts, this formula got its remarkable therapeutic effect in which more than 300 people—number is increasing—tried it without any noticeable side effects.

It is highly recommended to keep it in the fridge after opening because it freshly prepared without any chemical additions.

It is an effective treatment and its relieving results appears on the treatment of Irritable, inflammatory Bowel Syndrome and Gastritis. In most cases, the disorders' symptoms disappear for two years after completion of one treatment course (½ KG, one spoon twice a day). Furthermore, it has showed an incredible effect on Gastroesophageal reflux disease, where it is not only give a temporary soothing, but immediately begin to tighten the relaxation of the esophagus and stomach and treat the inflammatory, infections, and acid reflux symptoms. This was successfully approved by 90% of the patients treated with this formula. In some cases the digestive problems returned after 6 months because the patients were smokers.

It also succeeded in preventing the eradication of part of the stomach operation because of the semi-cancerous lesions in the stomach of one patient. Just a month after the use of treatment; 70% of these ulcers were disappeared and the operation was canceled. More than 90% who used Hadeem for stomach infections disclosed the real effective and affordable therapeutic effects of treatment. Finally, this formula has proven its usefulness in treating the vagaries of the digestive organs such as diarrhea and constipation.

It is NOT necessary to use the treatment for long-time and for maintained time, but it has a long-term effect can last beyond one to two years. The majority of cases did not have to request it more than once because all of the digestive problems were disappeared.

The treatment is a long-term treatment and not only a painkiller with knowing that it is completely has no side effects or negative health effects. Taking it from time to time for maintaining the health of the digestive system is advised

It is consists of raw organic components and it should be dried to avoid any un-desired reactions between the ingredients because of the humidity. Specific amounts of each ingredients should be carefully weighed and milled separately, then mixed well with honey and packed. After packing, it is completely not allowed to open the package and exposing it to the room air for long time to avoid microbial contamination. After opening, it is highly required to store in 5° C. and used within two months. Never keep it in room temperature. The Ingredients are: Organic Sedra Honey, grinded nutmeg, grinded flaxseed, grinded black seed. These ingredients grinded and mixed very well then kept tightly closed. These well-known components have no complications nor side effects as they are consumed daily in our meals. However, it has not been recorded that anyone has prepared this specific mixture for medical purposes and treating gastrointestinal problems.

For better results, it is recommended to take it ½ to 1 hour before the meals. Currently, it is consumed using a spoon like any regular food because it did not enter into the pharmaceutical industry on a commercial basis. Once it got licensed, it is planned to contract with pharmaceutical factory and manufactured in the form of capsules (1000 mg glycerol cover) and consumed three times a day. This is important to convince patients who do not like to the taste of mixture, although the treatment is not bitterness nor hot.

1- A natural formula for treating digestive problems and infections, and contains the following components:
a. Organic Sedra Honey
b. Black Seed
c. Linseed
d. Nutmeg

2- Composition according to the protection element number (1): consist of 70% of Organic Sedra honey.

3- Composition according to the protection element number (1): consist of 18% of black seed

4- Composition according to the protection element number (1): consist of 14% of Linseed

5- Composition according to the protection element number (1): consist of 8% of Nutmeg

6- Composition according to the protection elements number (1-5): where component (a) is the incubator and is added as fresh fluid form without any changes on its properties.

7- Composition according to the protection elements of component (A): the components (b,c,d) are added dryly after being well crushed to a fine powder and mixed with component (a) until they are completely mixed and homogenized.

8- Composition according to the protection elements number (7): where they are directly eaten or taken in the form of medical capsules packed with formula.
